(19) United States
(12) Patent Application Publication
(10) Pub. No.: US 2024/0349703 A1
(43) Pub. Date: Oct. 24, 2024

(54) TEMPORAL AND SPATIAL PHOTOGENERATION OF OLFACTORY MOLECULESSPEC

(71) Applicant: SIGNIFY HOLDING B.V., EINDHOVEN (NL)

(72) Inventors: MARC ANDRE DE SAMBER, LOMMEL (BE); DRAGAN SEKULOVSKI, EINDHOVEN (NL); HARRY BROERS, 'S-HERTOGENBOSCH (NL)

(21) Appl. No.: 18/687,381

(22) PCT Filed: Aug. 25, 2022
(86) PCT No.: PCT/EP2022/073706

§ 371 Date: Feb. 28, 2024

(30) Foreign Application Priority Data
Aug. 31, 2021 (EP) 21194025.9

(51) Int. Cl. A01K 29/00; A01K 1/00; A61M 21/00; A61M 21/02

(52) U.S. Cl. CPC A01K 29/005; A01K 1/0082; A61M 21/02; A61M 2021/0016; A61M 2250/00

(57) ABSTRACT

The invention provides a system (1000) for providing olfactory molecules (1220) in an animal residence (200), the system (1000) comprising (a) a radiation system (1100), (b) a precursor material providing system (1200), and (c) a control system (300), wherein: the radiation system (1100) is configured to generate first radiation (1111); the precursor material providing system (1200) is configured to provide precursor material 5 (1210). wherein the precursor material (1210) is configured to be at least partly converted by the first radiation (1111) into the olfactory molecules (1220); and the control system (300) is configured to control generation of the olfactory molecules (1220) by controlling one or more of the radiation system (1100) and the precursor material providing system (1200).

### FIELD OF THE INVENTION

The invention relates to a system for providing olfactory molecules. The invention also relates to an animal residence system comprising such system for providing olfactory molecules. Yet, the invention also relates to a method for controlling of animals in an animal residence.

### BACKGROUND OF THE INVENTION

Systems to control animals are known in the art. CA2383584C, for instance, describes a method for controlling breeding of animals of a herd within a breeding system, the method comprising for each animal the repeated series of steps of automatic identification of an individual animal by means of identification means controlled by means of a control unit to which an output comprising the identity of the animal is communicated from the identification means, dispersing feed to the identified animal by means of feed dispersing means of an automatic feeding unit controlled by means of the control unit, and adding data concerning the feeding of the identified animal to a record pertaining to said animal and storing said record on data storage means associated with the control unit, the data concerning the feeding of the identified animal comprises data for unique identification of the batch/batches of feed from which the feed dispersed to the animal is taken, wherein the step of monitoring characteristics of each identified animal comprises the monitoring of a temperature of the animal, being detected by suitable detection means controlled by means of the control unit to which the detection means communicates an output signal being significant for the detected characteristics, the method further comprising the step of adding said output from the detection; wherein a temperature of each animal is monitored, the method further comprising the steps of determining whether the detected temperature deviates from a predetermined reference temperature range by means of the control unit, and separating the animal by means of a separation unit controlled by means of the control unit in case of deviation.

### SUMMARY OF THE INVENTION

The ability to detect chemical stimuli is a primitive sensory capability that is found in nearly every animal species. And olfaction (detection of smell) is a ubiquitous sensory system found in all vertebrates, with functionalities in reproduction, physiology and in a large number of behaviors. Birds use olfaction for several important activities such as feeding and mating. Similarly, many fish activities may (partially) depend on olfaction, such as mating, discriminating kin, locating food, and avoiding predators. And pigs are reputed to have excellent olfactory abilities (see their ability to detect items such as truffles on the basis of their sense of smell). And in long-nosed mammals such as horses, cattle and sheep, olfactory senses are likely to be well developed as well. Similarly, insects may communicate using pheromones, such as to attract mates, warn others of predators, and to find food. Of particular interest and use may be pheromones, secreted or excreted chemical factors that trigger a social response in members of the same species. Pheromones are chemicals capable of acting like hormones outside the body of the secreting individual, as to impact the behavior of the receiving individuals. It appears that organisms cannot quickly transmit pheromones over long distances or change from one chemical message to another.

In industrial stables, the smell senses of animals may be disrupted and blurred, because the natural scents may not be available indoor and because of possible abundant smell concentration because of dense animal population. Or else, the loss of certain smells may be due to ventilation. This may lead to a strong and cumbersome deviation from natural conditions, which might impact physiology and behaviors of the animals.

Hence, it appears to be desirable to use olfactory molecules to influence the behavior of animals in an animal residence. However, when using olfactory molecules, it may be desirable to provide these in a controlled way, for instance locally in an animal residence and/or at a desired time. Hence, it is an aspect of the invention to provide an alternative system and/or method for controlling animals in an animal residence, which preferably further at least partly obviates one or more of above-described drawbacks. The present invention may have as object to overcome or ameliorate at least one of the disadvantages of the prior art, or to provide a useful alternative.

Amongst others, it is herein suggested to artificially generate scent molecules (e.g., pheromones) in the animal living environment. This may be achieved by introducing precursor molecules. These precursor molecules can be transformed into olfactory molecules, such as scent molecules, when photochemically activated. Therefore, after introduction of the precursor molecules in the environment, the lighting system may in embodiments be used to convert the precursor molecules into scent molecules. Such conversion can thus be controlled locally and at selected times. Locally means that you can also target individual animals. Additionally, in embodiments also methods for removal of olfactory molecules, such as scent molecules, are suggested. This may provide the option to counteract a key disadvantage of natural pheromones: the lack of options for fast change-over from one message to another message (=replacing one pheromone by another).

Hence, in a first aspect, the invention may provide a system for providing olfactory molecules in an animal residence. Especially, the system may comprise (a) a radiation system and (b) a precursor material providing system. Further, especially the system may comprise (c) a control system. In embodiments, the radiation system may be configured to generate first radiation. Especially, in embodiments the precursor material providing system may be configured to provide precursor material. Especially, the precursor material may be configured to be at least partly converted by the first radiation into the olfactory molecules. Yet, especially in embodiments the control system may be configured to control generation of the olfactory molecules, especially by controlling one or more of the radiation system and the precursor material providing system. Hence, in specific embodiments the invention provides a system for providing olfactory molecules in an animal residence, the system comprising (a) a radiation system, (b) a precursor material providing system, and (c) a control system, wherein: (A) the radiation system is configured to generate first radiation; (B) the precursor material providing system is configured to provide precursor material, wherein the precursor material is configured to be at least partly converted by the first radiation into the olfactory molecules; and (C) the control system is configured to control generation of the olfactory molecules by controlling one or more of the radiation system and the precursor material providing system.

With such system it may be possible to provide olfactory molecules at a desired time or within a desired time period. It may also be possible to locally provide the olfactory molecules, e.g. by locally providing the light and/or locally providing the precursor material (or “olfactory molecule precursors”). It may in specific embodiments even be possible to provide the precursor material on the animals or on specific animals, and target specific animals with the first radiation. Hence, amongst others the invention may allow in embodiments a temporal and spatial photogeneration of olfactory molecules.

As indicated above, the system may especially be configured to provide olfactory molecules. The term “olfactory molecule” may herein especially refer to a molecule detectable by an olfactory system (of an animal). For instance, (most) terrestrial vertebrates may detect olfactory molecules via (olfactory epithelium in) their noses. Similarly, aquatic animals, such as fish, may detect olfactory molecules via (olfactory epithelium in) nasal cavities. The olfactory molecule may be consciously detected by the animal, such as in the case of olfactory molecules picked up as scents, or may be unconsciously detected by the animal, such as may be the case for certain pheromones. It will be clear to the person skilled in the art that different animals may differ in their ability to detect olfactory molecules, and may differ in the molecules that they consciously smell. The term “olfactory molecules” and similar terms may herein especially refer to molecules that may be detected by the olfactory system of the animal hosted in the animal residence. Hence, in embodiments, the olfactory molecules may comprise scent molecules (for the animal). In further embodiments, the olfactory molecules may comprise pheromones (for the animal). Further, as indicated above, the system is especially configured to provide olfactory molecules in an animal residence. Hence, the animal residence is not necessarily part of the system. However, the animal residence may be comprised by the animal residence system, which is further discussed below. Essentially all embodiments relevant for the system may also be relevant for the animal residence system. Further, essentially all embodiments relevant for the animal residence system, if not intrinsically associated with the animal residence, may also be relevant for the system. Here below, first the system is discussed in more detail.

The animal residence may especially be configured to host an animal, such as an animal selected from the group comprising insects, poultry, cattle, pigs and aquatic animals, especially insects, or especially one or more of poultry, cattle and pigs, or especially aquatic animals. Hence, in embodiments, the animal residence may be configured to host insects, poultry, cattle, pigs or aquatic animals, such as fish, especially insects, or especially one or more of poultry, cattle and pigs, or especially aquatic animals. In embodiments, the animal residence may (be configured to) host poultry. In embodiments, the animal residence may (be configured to) host cattle. In embodiments, the animal residence may (be configured to) host pigs. In embodiments, the animal residence may (be configured to) host aquatic animals. In further embodiments, the animal residence may be configured to host terrestrial animals.

The system may especially comprise a radiation system and a precursor material providing system.

In or for animal residences, lighting systems may be available. However, the (visible) radiation of such systems may (in general) not provide the radiation that may be used herein for generating the olfactory molecules from precursor material. Especially, such radiation may be UV radiation, though other embodiments are herein not excluded. The fact that other radiation may be necessary to generate the olfactory molecules from precursor material, also facilitates the spatial and/or temporal control of the generation of the olfactory molecules from precursor material. Hence, a lighting system available in or for the animal may be adapted for use for the present invention, or a separate radiation system may be provided.

The radiation used to generate the olfactory molecules from precursor material is herein indicated as first radiation. Hence, the first radiation may especially be selected to (be suitable to) convert the precursor material into the olfactory molecules. Especially, in embodiments, the first radiation may (thus) be selected from UV radiation, i.e. radiation having one or more wavelengths in the UV, especially having one or more wavelengths in the 190-380 nm range, such as in the 200-380 nm range. However, wavelengths smaller than 190 nm are herein not excluded, and e.g. the first radiation may alternatively or additionally also have wavelength within the wavelength range of 100-190 nm range. Dependent upon the wavelengths, the first radiation may be applied for shorter or longer periods, or in specific way, such that exposure of the animal to the first radiation may be limited in time and intensity.

It may also be possible to use upconversion. Hence, precursor material may be used that can be converted into the olfactory molecules via an upconversion process, such as frequency doubling. In specific embodiments, it may be possible to use two or more light sources, which are focused at a same spatial position, such that a two-photon or other multi-photon process may be applied. In such embodiments, the radiation may thus be visible light. Especially, however, this may be executed with lasers.

Hence, for the radiation system for generation of the olfactory molecules from precursor material may apply in embodiments that the radiation system is configured to generate UV radiation and/or may apply in embodiments that the radiation system comprises laser light sources which are configurable or configured to generate at a same spatial position laser light of two or more laser light sources, allowing a multi-photon process. The two or more photons may be in the visible, i.e. having a wavelength selected from the range of 380-780 nm, or even one or more of them may have a wavelength selected from the IR range, such as e.g. be selected from the range of 780-1200 nm. For instance, using n lasers and focusing the laser in a point in the space of the animal residence may lead to an effective wavelength of 1/Σ(1/λₙ). For instance, a laser with 400 nm and a laser with 500 nm peak wavelengths may provide a wavelength a point in space where both lasers are focused of 222.2 nm.

The radiation system may (thus) comprise one or more light sources (see also below).

The phrase “converting the precursor material into the olfactory molecules using first radiation” and similar phrases may herein refer to a direct conversion of the precursor material into the olfactory molecules, but also to an indirect conversion. For instance, in embodiments, the precursor material, when exposed to the first radiation, may be converted into an intermediate molecule, which intermediate molecules may (spontaneously) convert, such as via isomerization, to the olfactory molecule. Similarly, in embodiments, the precursor material may spontaneously convert to the intermediate molecule, such as via a rearrangement reaction, wherein the intermediate molecule converts to the olfactory molecule when exposed to the first radiation. In further embodiments, the precursor material may convert to the intermediate molecule when exposed to (a first wavelength of) first radiation, and the intermediate molecule may convert to the olfactory molecule when exposed to (a second wavelength of) first radiation. In further embodiments, the first radiation may comprise (at least) two different wavelengths, wherein the olfactory molecule may be provided from the precursor material via two (or more) photoreactions: precursor+(first wavelength of) first radiation÷intermediate molecule; intermediate molecule+(second wavelength of) first radiation÷olfactory molecule. In particular, the first wavelength may result in the intermediate molecule, which might, for example, be unstable and might return back to the precursor molecule, thus successively requiring the second wavelength for stable formation of the olfactory molecule. However, in embodiments, the intermediate molecule, especially an intermediate pheromone, might also already have a certain (olfactory) functionality, wherein the second wavelength converts the intermediate molecule to the olfactory molecule, especially a pheromone.

Hence, in embodiments, the precursor material may be converted to olfactory molecules via one or more (photo-) chemical reactions, especially via a single chemical reaction, or especially via two or more chemical reactions. In particular, the one or more chemical reactions may comprise at least one photochemical reaction, such as at least two photochemical reactions, especially wherein the (or “each”) photochemical reaction comprises a conversion caused by the first radiation, such as by absorption of a (first) wavelength of the first radiation.

Hence, in embodiments, the olfactory molecules may be formed from the precursor material via an intermediate molecule, especially via a plurality of intermediate molecules. In further embodiments, the first radiation may comprise a first wavelength and a second wavelength, wherein the precursor material is converted into an intermediate molecule when exposed to the first wavelength, and wherein the intermediate molecule is converted into the olfactory molecule when exposed to the second wavelength.

The term “light source” may in principle relate to any light source known in the art. It may be a conventional (tungsten) light bulb, a low pressure mercury lamp, a high pressure mercury lamp, a fluorescent lamp, a LED (light emissive diode). In a specific embodiment, the light source comprises a solid state LED light source (such as a LED or laser diode (or “diode laser”)). The term “light source” may also relate to a plurality of light sources, such as 2-200 (solid state) LED light sources. Hence, the term LED may also refer to a plurality of LEDs. Further, the term “light source” may in embodiments also refer to a so-called chips-on-board (COB) light source. The term “COB” especially refers to LED chips in the form of a semiconductor chip that is neither encased nor connected but directly mounted onto a substrate, such as a PCB. Hence, a plurality of light emitting semiconductor light source may be configured on the same substrate. In embodiments, a COB is a multi LED chip configured together as a single lighting module.

The light source has a light escape surface. Referring to conventional light sources such as light bulbs or fluorescent lamps, it may be outer surface of the glass or quartz envelope. For LED's it may for instance be the LED die, or when a resin is applied to the LED die, the outer surface of the resin. In principle, it may also be the terminal end of a fiber. The term escape surface especially relates to that part of the light source, where the light actually leaves or escapes from the light source. The light source is configured to provide a beam of light. This beam of light (thus) escapes from the light exit surface of the light source.

The term “light source” may refer to a semiconductor light-emitting device, such as a light emitting diode (LEDs), a resonant cavity light emitting diode (RCLED), a vertical cavity laser diode (VCSELs), an edge emitting laser, etc. . . . The term “light source” may also refer to an organic light-emitting diode (OLED), such as a passive-matrix (PMOLED) or an active-matrix (AMOLED). In a specific embodiment, the light source comprises a solid-state light source (such as a LED or laser diode). In an embodiment, the light source comprises a LED (light emitting diode). The terms “light source” or “solid state light source” may also refer to a superluminescent diode (SLED).

The term LED may also refer to a plurality of LEDs. Further, the term “light source” may in embodiments also refer to a so-called chips-on-board (COB) light source. The term “COB” especially refers to LED chips in the form of a semiconductor chip that is neither encased nor connected but directly mounted onto a substrate, such as a PCB. Hence, a plurality of semiconductor light sources may be configured on the same substrate. In embodiments, a COB is a multi-LED chip configured together as a single lighting module.

The term “light source” may also relate to a plurality of (essentially identical (or different)) light sources, such as 2-2000 solid state light sources. In embodiments, the light source may comprise one or more micro-optical elements (array of micro lenses) downstream of a single solid-state light source, such as a LED, or downstream of a plurality of solid-state light sources (i.e. e.g. shared by multiple LEDs). In embodiments, the light source may comprise a LED with on-chip optics. In embodiments, the light source comprises a pixelated single LEDs (with or without optics) (offering in embodiments on-chip beam steering).

In embodiments, the light source may be configured to provide primary radiation, which is used as such, such as e.g. a blue light source, like a blue LED, or a green light source, such as a green LED, and a red light source, such as a red LED. Such LEDs, which may not comprise a luminescent material (“phosphor”) may be indicated as direct color LEDs.

In other embodiments, however, the light source may be configured to provide primary radiation and part of the primary radiation is converted into secondary radiation. Secondary radiation may be based on conversion by a luminescent material. The secondary radiation may therefore also be indicated as luminescent material radiation. The luminescent material may in embodiments be comprised by the light source, such as a LED with a luminescent material layer or dome comprising luminescent material. Such LEDs may be indicated as phosphor converted LEDs or PC LEDs (phosphor converted LEDs). In other embodiments, the luminescent material may be configured at some distance (“remote”) from the light source, such as a LED with a luminescent material layer not in physical contact with a die of the LED. Hence, in specific embodiments the light source may be a light source that during operation emits at least light at a wavelength selected from the range of 380-470 nm. However, other wavelengths may also be possible. This light may partially be used by the luminescent material.

A light source may be comprised by a light generating device. A light generating device may comprise a light source. A light generating device may also comprise one or more light sources. A light generating device may also comprise one or more light sources and optics, e.g. to beam shape the (light source) light of the one or more light sources.

In embodiments, the light generating device may comprise a luminescent material. In embodiments, the light generating device may comprise a PC LED. In other embodiments, the light generating device may comprise a direct LED (i.e. no phosphor). In embodiments, the light generating device may comprise a laser device, like a laser diode. In embodiments, the light generating device may comprise a superluminescent diode. Hence, in specific embodiments, the light source may be selected from the group of laser diodes and superluminescent diodes. In other embodiments, the light source may comprise an LED.

The light source may especially be configured to generate light source light having an optical axis (O), (a beam shape,) and a spectral power distribution. The light source light may in embodiments comprise one or more bands, having band widths as known for lasers

The term “light source” may (thus) refer to a light generating element as such, like e.g. a solid state light source, or e.g. to a package of the light generating element, such as a solid state light source, and one or more of a luminescent material comprising element and (other) optics, like a lens, a collimator. A light converter element (“converter element” or “converter”) may comprise a luminescent material comprising element. For instance, a solid state light as such, like a blue LED, is a light source. A combination of a solid state light source (as light generating element) and a light converter element, such as a blue LED and a light converter element, optically coupled to the solid state light source, may also be a light source. Hence, a white LED is a light source.

The term “light source” herein may also refer to a light source comprising a solid state light source, such as an LED or a laser diode or a superluminescent diode. The “term light source” may (thus) in embodiments also refer to a light source that is (also) based on conversion of light, such as a light source in combination with a luminescent converter material. Hence, the term “light source” may also refer to a combination of a LED with a luminescent material configured to convert at least part of the LED radiation, or to a combination of a (diode) laser with a luminescent material configured to convert at least part of the (diode) laser radiation. In embodiments, the term “light source” may also refer to a combination of a light generating device, like a LED, and an optical filter, which may change the spectral power distribution of the light generated by the light generating device.

The phrases “different light sources” or “a plurality of different light sources”, and similar phrases, may in embodiments refer to a plurality of solid-state light sources selected from at least two different bins. Likewise, the phrases “identical light sources” or “a plurality of same light sources”, and similar phrases, may in embodiments refer to a plurality of solid-state light sources selected from the same bin.

The term “solid state light source”, and similar terms, may especially refer to semiconductor light sources, such as a light emitting diode (LED), a diode laser, or a superluminescent diode.

The term “laser light source” especially refers to a laser. Such laser may especially be configured to generate laser light source light having one or more wavelengths in the UV, visible, or infrared, especially having a wavelength selected from the spectral wavelength range of 200-2000 nm, such as 300-1500 nm. The term “laser” especially refers to a device that emits light through a process of optical amplification based on the stimulated emission of electromagnetic radiation.

Especially, in embodiments the term “laser” may refer to a solid-state laser. In specific embodiments, the terms “laser” or “laser light source”, or similar terms, refer to a laser diode (or diode laser).

Hence, in embodiments the light source comprises a laser light source. In embodiments, the terms “laser” or “solid state laser” may refer to one or more of cerium doped lithium strontium (or calcium) aluminum fluoride (Ce:LiSAF, Ce:LiCAF), chromium doped chrysoberyl (alexandrite) laser, chromium ZnSe (Cr:ZnSe) laser, divalent samarium doped calcium fluoride (Sm:CaF₂) laser, Er:YAG laser, erbium doped and erbium-ytterbium codoped glass lasers, F-Center laser, holmium YAG (Ho:YAG) laser, Nd:YAG laser, NdCrYAG laser, neodymium doped yttrium calcium oxoborate Nd:YCa₄O(BO₃)₃ or Nd:YCOB, neodymium doped yttrium orthovanadate (Nd:YVO₄) laser, neodymium glass (Nd:glass) laser, neodymium YLF (Nd:YLF) solid-state laser, promethium 147 doped phosphate glass (147Pm³⁺:glass) solid-state laser, ruby laser (Al₂O₃:Cr³⁺), thulium YAG (Tm:YAG) laser, titanium sapphire (Ti:sapphire; Al₂O₃:Ti³⁺) laser, trivalent uranium doped calcium fluoride (U:CaF₂) solid-state laser, Ytterbium doped glass laser (rod, plate/chip, and fiber), Ytterbium YAG (Yb:YAG) laser, Yb₂O₃ (glass or ceramics) laser, etc.

For instance, including second and third harmonic generation embodiments, the light source may comprise one or more of an F center laser, a yttrium orthovanadate (Nd:YVO₄) laser, a promethium 147 doped phosphate glass (147Pm³⁺:glass), and a titanium sapphire (Ti:sapphire; Al₂O₃:T³⁺) laser. For instance, considering second and third harmonic generation, such light sources may be used to generated blue light.

In embodiments, the terms “laser” or “solid state laser” may refer to one or more of a semiconductor laser diodes, such as GaN, InGaN, AlGaInP, AlGaAs, InGaAsP, lead salt, vertical cavity surface emitting laser (VCSEL), quantum cascade laser, hybrid silicon laser, etc.

A laser may be combined with an upconverter in order to arrive at shorter (laser) wavelengths. For instance, with some (trivalent) rare earth ions upconversion may be obtained or with non-linear crystals upconversion can be obtained. Alternatively, a laser can be combined with a downconverter, such as a dye laser, to arrive at longer (laser) wavelengths.

As can be derived from the below, the term “laser light source” may also refer to a plurality of (different or identical) laser light sources. In specific embodiments, the term “laser light source” may refer to a plurality N of (identical) laser light sources. In embodiments, N=2, or more. In specific embodiments, N may be at least 5, such as especially at least 8. In this way, a higher brightness may be obtained. In embodiments, laser light sources may be arranged in a laser bank (see also above). The laser bank may in embodiments comprise heat sinking and/or optics e.g. a lens to collimate the laser light.

The laser light source is configured to generate laser light source light (or “laser light”). The light source light may essentially consist of the laser light source light. The light source light may also comprise laser light source light of two or more (different or identical) laser light sources. For instance, the laser light source light of two or more (different or identical) laser light sources may be coupled into a light guide, to provide a single beam of light comprising the laser light source light of the two or more (different or identical) laser light sources. In specific embodiments, the light source light is thus especially collimated light source light. In yet further embodiments, the light source light is especially (collimated) laser light source light.

The laser light source light may in embodiments comprise one or more bands, having band widths as known for lasers. In specific embodiments, the band(s) may be relatively sharp line(s), such as having full width half maximum (FWHM) in the range of less than 20 nm at RT, such as equal to or less than 10 nm. Hence, the light source light has a spectral power distribution (intensity on an energy scale as function of the wavelength) which may comprise one or more (narrow) bands.

The beams (of light source light) may be focused or collimated beams of (laser) light source light. The term “focused” may especially refer to converging to a small spot. This small spot may be at the discrete converter region, or (slightly) upstream thereof or (slightly) downstream thereof. Especially, focusing and/or collimation may be such that the cross-sectional shape (perpendicular to the optical axis) of the beam at the discrete converter region (at the side face) is essentially not larger than the cross-section shape (perpendicular to the optical axis) of the discrete converter region (where the light source light irradiates the discrete converter region). Focusing may be executed with one or more optics, like (focusing) lenses. Especially, two lenses may be applied to focus the laser light source light. Collimation may be executed with one or more (other) optics, like collimation elements, such as lenses and/or parabolic mirrors. In embodiments, the beam of (laser) light source light may be relatively highly collimated, such as in embodiments ≤2° (FWHM), more especially ≤1° (FWHM), most especially ≤0.5° (FWHM). Hence, ≤2° (FWHM) may be considered (highly) collimated light source light. Optics may be used to provide (high) collimation (see also above).

In specific embodiments, the radiation system may comprise a plurality of different light sources, such as two or more subsets of light sources, with each subset comprising one or more light sources configured to generate light source light having essentially the same spectral power distribution, but wherein light sources of different subsets are configured to generate light source light having different spectral distributions. In such embodiments, a control system may be configured to control the plurality of light sources. In specific embodiments, the control system may control the subsets of light sources individually.

Hence, the radiation system may especially comprise one or more semiconductor light sources, such as one or more light emitting diodes (LED), one or more diode lasers, and/or one or more superluminescent diodes.

Hence, the radiation system may especially be configured to generate in an operational mode first radiation. In specific embodiments, this first radiation may be UV radiation, like UV-C radiation. For instance, in embodiments the first radiation may comprise one or more of UV-A (315-380 nm), UV-B (280-315 nm), Near UV-C (230-280 nm), Far UV-C (190-230 nm), and Extreme UV-C (100-190 nm), especially one or of UV-A (315-380 nm), UV-B (280-315 nm), Near UV-C (230-280 nm), Far UV-C (190-230 nm), such as one or more of Near UV-C (230-280 nm) and Far UV-C (190-230 nm). Especially, in embodiments the first radiation may have a centroide wavelength selected from one of the following wavelength ranges UV-A (315-380 nm), UV-B (280-315 nm), Near UV-C (230-280 nm), Far UV-C (190-230 nm), and Extreme UV-C (100-190 nm), especially one of UV-A (315-380 nm), UV-B (280-315 nm), Near UV-C (230-280 nm), Far UV-C (190-230 nm), such as one of Near UV-C (230-280 nm) and Far UV-C (190-230 nm). Hence, in embodiments the radiation system is configured to generate in a first operational mode (of the radiation system) the first radiation.

In specific embodiments, the radiation system may also be configured to provide (in an operational mode) second radiation, e.g. for general lighting or target lighting for the specific animal(s) in the animal residence. This may especially be visible radiation, which may be beneficial for the animals and/or for vision of a human, such as a farmer. Hence, in embodiments the radiation system may be configured to generate in a second operational mode (of the radiation system) second radiation, wherein especially the second radiation comprises visible radiation. Hence, in specific embodiments the radiation system may also be indicated as “light generation system” or “light generating system”. Therefore, in specific embodiments the radiation system may be configured to generate in a first operational mode (of the radiation system) the first radiation and in a second operational mode (of the radiation system) second radiation, wherein the first radiation and the second radiation have different spectral power distributions; especially wherein the first radiation comprises UV radiation and wherein the second radiation comprises visible radiation.

Further, as indicated above, the system may comprise the precursor material providing system. The precursor material providing system may be configured to provide precursor material, especially to (an indoor space of) the animal residence, or especially to an animal in the animal residence. The precursor material providing system may be configured to spread the precursor material through the (indoor space of) the animal residence. However, the precursor material providing system may further be configured to apply the precursor material at one or more specific sites in the animal residence. Yet further, the precursor material providing system may be a passive system, especially a passive system configured to release the precursor material upon receiving an external stimulant, such as upon being exposed to the first radiation.

Hence, in embodiments, the precursor material providing system may comprise one or more of a (i) nebulizer system, (ii) a spray system, (iii) a dripping system, (iv) a first radiation accessible precursor material reservoir, (v) an applicator system, and (vi) a fluid flow control system.

In particular, in embodiments, the precursor material providing system may comprise one or more of a (i) nebulizer system, (ii) a spray system, and (iii) a dripping system, especially the nebulizer system, or especially the spray system, or especially the dripping system. Such systems, especially the nebulizer and spray systems, may be particularly suitable for spreading the precursor material throughout the (indoor space of the) animal residence. In particular, in such embodiments, the precursor material may be (homogeneously) distributed throughout the (indoor space of the) animal residence, and the control system may be configured to control the radiation system for spatio-temporal control of the generation of the olfactory molecules.

In further embodiments, the precursor material providing system may comprise the first radiation accessible precursor material reservoir. In such embodiments, the precursor material providing system may especially be configured to passively release the precursor material upon receiving an external stimulant, such as upon being exposed to the first radiation. Such embodiments may be particularly suitable for (providing) olfactory molecules at specific predetermined sites, as well as for precursor material that is to be sparsely used, such as for precursor material with (detrimental) side effects, or such as for (relatively) expensive precursor material. In further embodiments, the first radiation accessible precursor material reservoir may comprise one or more of (i) a liquid container, such as a trough-like system, (ii) a coated material, such as a coated tray, (iii) a vesicle, and (iv) a sticker.

In further embodiments, the precursor material and the radiation system may be configured to release the olfactory molecules from the precursor material by irradiating the precursor material with the first radiation. For instance, the olfactory molecules may be arranged in a container, such as in a shell, or such as in a vesicle, which may (irreversibly) open when exposed to the first radiation, such as via a photophysical process.

In further embodiments, the precursor material providing system may comprise the applicator system. The applicator system may especially be configured to apply the precursor material on a surface, such as onto the skin of an animal. Hence, in embodiments, the applicator system may be configured for (targeted) application of the precursor material on an animal. For instance, in embodiments, the applicator system may comprise a livestock (comfort) brush, and may be configured to apply the precursor material to the animal while/by brushing the animal. In further embodiments, the applicator system may comprise a remote applicator element, such as a remote applicator gun, configured to apply the precursor material to the surface remotely, akin to providing (photoreactive) paint to the surface using a paint ball gun.

In further embodiments, the applicator system may comprise a fluid flow control system. The fluid flow control system may especially be configured to control the flow of a fluid, especially a gas, or especially a liquid, into (and/or out of) the (indoor space of the) animal residence. In particular, the fluid flow control system may comprise or be functionally coupled to a fluid reservoir, wherein the fluid reservoir comprises a fluid, especially a gas, or especially a liquid, comprising the precursor material, and wherein the fluid flow control system is configured to move the fluid from the fluid reservoir to the (indoor space of) the animal residence. Such embodiments may be particularly suitable for providing the precursor material in an aquacultural setting, such as in an animal residence configured for the hosting of fish, or such as in an animal residence configured for the hosting of crustaceans.

As will be clear to the person skilled in the art, also combinations of two or more of the above mentioned precursor material providing systems may be employed. For instance, in embodiments, the applicator system may comprise a combined spray and applicator system, configured to spray the precursor material onto a surface, such as onto the skin of an animal.

The term “precursor material” may herein also refer to a plurality of different precursor materials, such as precursor materials that may be converted into respective different olfactory molecules. In particular, in such embodiments, the different precursor materials may be converted into respective different olfactory molecules upon exposure to different (wavelengths of) first radiation.

As described above, the precursor material may be configured to be at least partly converted by the first radiation into the olfactory molecules, i.e., upon exposure to the first radiation, the precursor material may (at least partly) be converted into the olfactory molecule. In particular, the precursor material may (be configured to) undergo a photochemical reaction upon exposure to the first radiation. Optionally, the photochemical reaction may involve one or more other precursor compounds, such as O₂, N₂, CO₂, and H₂O, or such as one or more other precursor compounds specifically introduced into the (indoor space of the) animal residence. Similarly, the photochemical reaction may produce one or more by-products, i.e., the precursor material (and optionally one or more other precursor compounds) may be converted into the olfactory molecules and, optionally, one or more by-products. Further, as will be clear to the person skilled in the art, the precursor material may, depending on the material and (environmental) conditions, be partly converted to other compounds, either in the presence or absence of the first radiation.

The invention is not limited to any specific precursor material or any specific photochemical reaction. For instance, in embodiments, the photochemical reaction may comprise one or more of an electrocyclic reaction, a radical reaction, a photoisomerization, and a Norrish reaction.

The precursor material may especially be selected in order to provide a specific olfactory molecule. For instance, the chemical group of fatty acids may comprise powerful pheromones and can be formed by, e.g., a photolysis reaction starting from saturated hydrocarbon precursors. As a further example, olfactory-active aromatic alkene configurations might be formed by a photochemical cis-trans isomerization conversion.

Hence, in embodiments, the precursor material may comprise a saturated hydrocarbon. In such embodiments, the olfactory molecule may especially comprise a fatty acid. In further embodiments, the precursor material may comprise an aromatic alkene. In further embodiments, the olfactory molecule may comprise an aromatic alkene.

In particular, the olfactory molecule may be selected to elicit a response in the animal (hosted in the animal residence). For instance, in embodiments, the olfactory molecule may (be configured to) affect an animal behavior (of the animal) selected from the group comprising stress, aggression, mating, feeding, sleeping, and moving.

For instance, the olfactory molecules 4-methylpentanal and hexanal may be anxiety pheromones for rats, and may, thus, be used to affect, for instance, stress in rats. Androstenone (5α-androst-16-en-3-one) is a known steroidal pheromone for swine. It may be found in boar's saliva and truffle fungus. Androstenone was the first mammalian pheromone to be identified. It may be found in high concentrations in the saliva of male pigs, and, when inhaled by a female pig that is in heat, may result in the female assuming the mating stance. Hence, androstenone may, for instance, be used to promote mating behavior. Similarly, aromatic alkenes may be known as pheromones for cattle, may be present in animal secretions (perspiration, urine, faeces, oestrus and vaginal secretions), may play a role in social organization in the group and in recognition of individual animals, and may modulate reproduction.

Further, scented molecules with (for the animal) a foul smell may be used to control movement of the animal, such as to keep an animal out of an indoor space, or such as to coerce an animal to leave an indoor space.

In embodiments, the animal residence, especially the indoor space, may comprise a catalyst configured to catalyze the photochemical reaction. For instance, in embodiments, the photochemical reaction may comprise a photochemical oxidation, and the catalyst may be configured to catalyze the photochemical oxidation. Hence, in embodiments, the catalyst may, for example, comprise TiO₂. In particular, in such embodiments, radicals may be formed via the reaction: H₂O+TiO₂+first radiation (UVA; e.g. 360 nm)→OH (radical)+TiO₂ (TiO₂ is not consumed). In particular, in such embodiments, the OH radical may react with the precursor material to provide the olfactory molecules, i.e., the OH radical may next be the chemical reagent for the conversion of the precursor molecule into the olfactory molecule. For example, fatty acids can be formed this way starting from an aldehyde precursor.

Hence, in embodiments, the precursor material may comprise an aldehyde. In such embodiments, the olfactory molecule may especially comprise a fatty acid.

In further embodiments, the precursor material may be configured to absorb (a first wavelength of) the first radiation, especially wherein the precursor material is converted to the olfactory molecules following absorption of the (first wavelength of the) first radiation.

Hence, in embodiments, the precursor material and the radiation system may be configured to provide the olfactory molecules by converting at least part of precursor material into the olfactory molecules by irradiating the precursor material with the first radiation.

In further embodiments, the (first wavelength of the) first radiation may be selected to be absorbed by the precursor material, especially to excite the precursor material.

Yet further, the system may comprise a control system. Especially, the control system may be configured to control generation of the olfactory molecules by controlling one or more of the radiation system and the precursor material providing system, especially by controlling the radiation system, or especially by controlling the precursor material providing system.

In particular, the control system may control the generation of the olfactory molecule in (an indoor space of) the animal residence. The generation of the olfactory molecule may depend on (or “be a result of”) the concurrent colocalization of the precursor material and the first radiation. Hence, the control system may, for instance, control the presence of the precursor material in the indoor space by controlling the precursor material providing system, or may control the presence of the first radiation by controlling the radiation system.

In particular, if the precursor material is converted to the olfactory molecules by visible first radiation, it may be particularly beneficial to control the generation of the olfactory molecule by controlling the precursor material providing system, so as to avoid the generation of the olfactory molecules by ambient light. In contrast, if the precursor material is converted to the olfactory molecules by, for instance, UV radiation, it may be particularly beneficial to control the generation of the olfactory molecules by controlling the radiation system, so as to limit the amount of UV radiation provided to the (indoor space of the) animal residence.

In embodiments wherein the precursor material providing system is configured to provide a plurality of different precursor materials, the control system may also be configured to control the generation of a first subset of olfactory molecules by controlling the presence of a corresponding first subset of precursor materials by controlling the precursor material providing system, and to control the generation of a second subset of olfactory molecules by controlling the radiation system.

As described above, in embodiments, the control system may be configured to control the precursor material providing system to control the providing of precursor material, especially to (the indoor space of) the animal residence. For instance, the control system may control the precursor material providing system to provide the precursor material to (essentially) directly convert the precursor material to the olfactory molecules, or, for instance, to replenish the precursor material (in the animal residence) following application of the first radiation.

In further embodiments, the precursor material providing system may be configured to (essentially) continuously provide the precursor material. In further embodiments, the precursor material providing system may be configured to periodically provide the precursor material. In a further aspect, the invention may provide an animal residence system comprising (i) an animal residence and (ii) the system according to the invention. As described above, the animal residence may especially be configured to host an animal, such as an animal selected from the group comprising insects, poultry, cattle, pigs, and aquatic animals. In embodiments, the animal residence may comprise an indoor space configured for hosting the animal, especially for hosting a plurality of (same) animals.

Hence, the system may especially be configured to control the generation of the olfactory molecules in the animal residence, such as in the indoor space of the animal residence, or especially in a plurality of different indoor spaces of the animal residence.

In embodiments, the radiation system may be configured to generate the first radiation in the animal residence, especially in the indoor space of the animal residence. In further embodiments, the radiation system may be configured to provide the first radiation at different locations in the animal residence, especially in a plurality of different indoor spaces. In particular, in further embodiments, the radiation system may comprise a plurality of first light sources configured to provide the first radiation at the different locations in the animal residence.

In further embodiments, the precursor material providing system may be configured to provide the precursor material in the animal residence, especially in the indoor space. In further embodiments, the precursor material providing system may be configured to provide the precursor material at different locations in the animal residence, especially in a plurality of different indoor spaces.

In further embodiments, the control system may be configured to control the generation of the olfactory molecules in the animal residence, especially in the indoor space, by controlling one or more of the radiation system and the precursor material providing system. In particular, in embodiments, the control system may be configured to control local generation of the olfactory molecules in the animal residence by controlling one or more of (i) the precursor material providing system and (ii) the radiation system.

As described above, in embodiments, the precursor material providing system (of the system) may comprise an applicator system. In particular, the applicator system may comprise one or more applicator system elements configured to apply the precursor material to an animal. In further embodiments, one or more of the following may apply: (i) a position of at least one applicator system element within the animal residence is controllable by the control system; (ii) the animal residence system comprises a plurality of applicator system elements configured at different locations within the animal residence and configured to locally apply the precursor material to the animal; and (iii) at least one applicator system element is configured at a location where the animal is present at (at least) a predetermined frequency, such as at least once a day, such as at a location selected from the group comprising a feeding place (or entrance thereto), a milking street, and a sleeping site.

For instance, in embodiments, the control system may be configured to control the location of the at least one applicator system element within the animal residence. In particular, the control system may be configured to control the location of the at least one application system element via one or more of a robot arm and a rail.

As described above, the precursor material providing system may be configured to provide a plurality of different precursor materials, which may be converted to a plurality of different olfactory molecules. Hence, in embodiments, the control system may be configured to control the type of olfactory molecules provided (to the animal residence). Similarly, the control system may be configured to control one or more of the rate at with which the olfactory molecules are provided, and/or an amount in which the olfactory molecules are provided. In particular, animals may be sensitive not only to the presence (or absence) of an olfactory molecule, but also to the rate at which the amount (or “concentration”) of the olfactory molecule changes over time, as well as to the absolute amount (or “concentration”) of the olfactory molecule.

The control system may, in embodiments, operate based on a predetermined program. For instance, the control system may be configured to coerce the animal(s) to move towards a feeding area at a predetermined time of day. Similarly, the control system may be configured to coerce the animal(s) to sleep at a (different) predetermined time of day. Hence, the control system may be configured to control the generation of the olfactory molecules, especially to control one or more of the radiation system and the precursor material providing system, based on a timer.

However, the control system may further be configured to control the generation of the olfactory molecules in dependence of one or more factors, such as animal behavior inside the animal residence, a user input, and a sensor signal.

Hence, in further embodiments, the control system may be configured to control one or more of (i) a rate at with which the olfactory molecules are provided, (ii) an amount in which the olfactory molecules are provided, and (iii) the type of olfactory molecules provided in dependence of one or more of (a) a user input, (b) a sensor signal of a sensor (arranged in the animal residence), and (c) a timer.

Hence, in embodiments, a sensor may be arranged in the animal residence. In particular, the animal residence may comprise a sensor. The sensor may especially be configured to determine a parameter in the animal residence and to provide a related signal to the control system. In such embodiments, the control system may be configured to control the radiation system and/or the precursor material providing system in dependence of the behavior signal.

The term “sensor” may herein also refer to a plurality of sensors, such as a plurality of same sensors arranged in different (sensor) locations, or such as a plurality of different sensors. Hence, the sensor may, in embodiments, comprise a sensor array.

In further embodiments, the sensor may be configured to determine an animal behavioral parameter in the animal residence, and to provide a related behavior signal to the control system. Hence, in embodiments, the parameter may comprise an animal behavioral parameter, and the related signal may comprise a related behavior signal. In further embodiments, the animal behavior signal may be selected from the group comprising moving, interacting (with other animals), sleeping, eating, mating, fighting, and vocalizing. In such embodiments, the control system may, for instance, control the (local) generation of the olfactory molecule to stimulate desired behavior and/or to discourage undesired behavior, such as aggressive behavior.

Similarly, in further embodiments, the sensor may be configured to determine an environmental parameter in the animal residence, and to provide a related environmental signal to the control system. Hence, in embodiments, the parameter may comprise an environmental parameter, and the related signal may comprise a related environmental signal. In further embodiments, the environmental parameter may be selected from the group comprising temperature, (relative) humidity, particulate matter, and noise.

In further embodiments, the sensor may be configured to determine an animal location parameter (of the animals), and to provide a related (location) signal to the control system. For example, in case of crowding at a specific location (too many animals gathering together, with a risk of suffocation or high stress), the control system may then control the radiation system and/or the precursor material providing system to provide a deterring condition with olfactory molecules at that specific location, leading to a spreading of the animals and a return to normal.

In further embodiments, the sensor, especially a chemical sensor, such as an electronic nose, may be configured to measure (a concentration of) the olfactory molecules, and to provide a related (olfaction) signal to the control system. Thereby, the control system may tune the (concentration of) the olfactory molecules, especially to control animal behavior. In particular, multiple factors may influence the generation and removal/degradation of the olfactory molecules. For instance, ambient light, air flow (in/out of the system), and natural production of olfactory molecules by animals, may all influence the (concentration of) olfactory molecules in the animal residence. Hence, it may be beneficial for the control system to have access to a measurement of the olfactory molecules in order to more appropriately tune the generation of the olfactory molecules to reach a desired concentration of the olfactory molecules.

The term “related signal”, such as “related behavior signal”, may herein refer to a signal that is related to the detected parameter, such as to the detected animal behavior. In particular, the related signal may comprise raw and/or processed data related to the (detected) parameter.

In embodiments, the sensor may comprise one or more (dedicated) sensors for measuring the animal behavioral parameter. For instance, the sensor element may comprise a (micro-electro-mechanical-systems; MEMS) accelerometer or a (MEMS) gyroscope to measure a positional parameter, especially acceleration, or especially a rate of turn sensing. Similarly, the sensor may comprise a heart rate monitor to measure a physiological parameter, especially the heart rate. It will be clear to the person skilled in the art that many types of sensors are possible within the scope of the invention.

In embodiments, the sensor may be configured to measure a physiological parameter, especially a physiological parameter selected from the group comprising a vital sign, such as heart rate, spO₂, and body temperature (as indicator for detecting an infection or high levels of activity), a hormonal parameter, such as fertility, which may be determined using gas detection or indirectly based on analysis of movements/behavior, and a skin electrical conductivity.

In further embodiments, the sensor element may be configured to measure a pathological parameter, especially a pathological parameter selected from the group comprising a microbial presence, fever, or (night) sweat.

In further embodiments, the sensor element may be configured to measure a animal behavioral parameter, especially an animal behavioral parameter selected from the group comprising movement (during the day) and interaction with other animals. In embodiments, the sensor element may comprise a proximity sensor (UWB, Zigbee/BLE, etc.) for measuring interactions with other animals.

In further embodiments, the sensor may be configured to measure an environmental parameter, especially an environmental parameter selected from the group comprising an ambient temperature, ambient noise, ambient air flow (or “draft”), and humidity.

In further embodiments, the sensor may be configured to measure a positional parameter, especially a positional parameter (of the animal) selected from the group comprising a location, a movement, a movement direction, and an acceleration. The positional parameter may, for instance, be indicative of where the animal is with respect to other animals or infrastructure. For example, it might be desired to provide actuation to motivate the animal to move out of an area if it has been there for too long (e.g., “hoarding” food for too long, or roaming outside too far from the rest of the herd).

For example, the sensor element may, in embodiments, measure a positional parameter, especially a location. The control system may be configured to, in case of a long period of observed inactivity of an animal, (locally) generate olfactory molecules to give the animal a ‘wake-up call’ to make the animal move again and show normal activity, as a signal that the animal is OK.

As the olfactory molecules may influence the behavior of the animal(s), the presence of the olfactory molecules may, at some timepoint, no longer be needed or even become detrimental. Hence, the animal residence system, especially the system, more especially the control system, may (also) be configured to remove the olfactory molecules, especially from the animal residence, such as from the indoor space.

For instance, in embodiments, the animal residence system, especially the system, may comprise a (air) ventilation system configured to replace air in the (indoor space of the) animal residence with (fresh) second air, wherein the second air is (essentially) devoid of the olfactory molecules.

In further embodiments, the radiation system may be configured to generate third radiation, wherein the third radiation is configured to remove the olfactory molecules, especially to modify the olfactory molecules to species that (i) are not olfactory, (ii) have a smaller olfactory effect, or (iii) have a different olfactory effect.

Further, as described above, the radiation system may (also) be configured to generate second radiation, especially wherein the second radiation comprises visible radiation. Hence, the radiation system may, in embodiments, providing lighting (via the second radiation), may facilitate generation of the olfactory molecules (via the first radiation), and may facilitate removal of the olfactory molecules (via the third generation). Hence, in embodiments, the first radiation, the second radiation, and the third radiation may have different spectral power distributions.

In particular, in embodiments, the radiation system may be configured to generate in a first operational mode (of the radiation system) the first radiation; to generate in a second operational mode (of the radiation system) the second radiation, to generate in a third operational mode (of the radiation system) the third radiation; wherein the first radiation, the second radiation, and the third radiation have different spectral power distributions. Especially, in further embodiments, the first radiation may comprise UV radiation, the second radiation may comprise visible radiation; and the third radiation may be configured to modify the olfactory molecules to species that are not olfactory, that have a smaller olfactory effect, or have a different olfactory effect.

The term “animal residence” may herein especially refer to any accommodation suitable for the keeping of animals, especially livestock. Generally, an animal residence may be any structure, especially (in) a building, configured to house livestock. The animal residence may comprise an indoor space. The indoor space may especially be configured for housing the animals, especially the livestock. In embodiments, the indoor space may comprise stalls. Additionally or alternatively, the indoor space may comprise (specialized) farm equipment such as feeding equipment and/or milking equipment. In embodiments, the indoor space may comprise a rearing space. In further embodiments, the animal residence may comprise one or more of a stable, a barn, a shed, and a pen.

Hence, in embodiments, the animal residence may be configured for the keeping of livestock, especially of pigs, or especially of cattle.

The term “livestock” (also: “animal”) may herein generally refer to any wild or domesticated animal raised in an agricultural setting to produce an animal product. The term “livestock” especially refers to any farm animal, such as cattle, including cows, sheep, goats, pigs, horses, fish and/or poultry. The term livestock may further refer to any animal that is kept providing an animal product, including animals such as worms and insects providing alternative protein sources. Livestock especially refers to ruminants, more especially to cattle, such as cows.

In embodiments, the system may comprise one or more actuators to further control the animals. For instance, the control system may be configured to control one or more of temperature, humidity, air flow, number of animals in the animal residence, etc. etc. . . . Hence, in addition to controlling the generation of the olfactory molecules, also (other) (environmental) parameters may be controlled (in dependence of the sensor signal). This control may e.g. be based on one or more of a feedback or feed forward control.

In embodiments, the control system may be configured to control one or more of the spectral power distribution of the first radiation, a duty cycle of the first radiation, a dynamic lighting effect of the first radiation, a spatial direction of (a first beam of) the first radiation, and the intensity of the first radiation, especially in dependence of the sensor signal. Hence, the control system may be configured to control one or more properties of the first radiation in dependence of the sensor signal. The control system may especially be configured to control the spectral power distribution of the first radiation in dependence of the sensor signal.

In embodiments, the system, especially the control system, may have an operational mode. The term “operational mode” may also be indicated as “controlling mode”. The system, or apparatus, or device (see further also below) may execute an action in a “mode” or “operational mode” or “mode of operation”. Likewise, in a method an action, stage, or step may be executed in a “mode” or “operation mode” or “mode of operation”. This does not exclude that the system, or apparatus, or device may also be adapted for providing another operational mode, or a plurality of other operational modes. Likewise, this does not exclude that before executing the mode and/or after executing the mode one or more other modes may be executed. However, in embodiments a control system may be available, that is adapted to provide at least the operational mode. Would other modes be available, the choice of such modes may especially be executed via a user interface, though other options, like executing a mode in dependence of a sensor signal or a (time) scheme, may also be possible. The operational mode may in embodiments also refer to a system, or apparatus, or device, that can only operate in a single operation mode (i.e. “on”, without further tunability).

In embodiments, the precursor material providing system may comprise a cartridge holder. The cartridge holder may especially be configured to detachably host one or more cartridges, especially a plurality of cartridges. In embodiments, the cartridge holder may be configured to host at least a cartridge comprising (at least part of) the precursor material. In further embodiments, the cartridge holder may be configured to host a plurality of cartridges comprising (different) precursor materials.

In specific embodiments, the cartridge holder may be configured to host a plurality of cartridges, wherein two or more of the plurality of cartridges are filled with different precursor materials

In further embodiments, the system may comprise a cartridge sensor configured to determine a remaining capacity (or volume) of precursor material in cartridges in the precursor material providing system.

Hence, the precursor material providing system may comprise (cartridges comprising) different types of precursor material. In embodiments, the control system may be configured to control the precursor material providing system to emit (part of) the precursor material based on a current (determined/estimated) amount of precursor material in the animal residence.

In further embodiments, the radiation system may especially comprise an upper air device configured to provide first radiation to the ceiling and/or a (top) part of a wall. Such embodiments may be particularly beneficial in embodiments wherein the first radiation comprises a wavelength (potentially) detrimental to the animal and/or to a human, such as for first radiation comprising UV radiation.

In a further aspect, the invention may provide a method for controlling animals in an animal residence. The method may comprise providing precursor material in the animal residence, especially in an indoor space of the animal residence. Especially, the precursor material may be configured to be at least partly converted by first radiation into olfactory molecules, wherein the olfactory molecules are olfactory molecules for one or more of the animals in the animal residence. The method may further comprise controlling the generation of the olfactory molecules by controlling one or more of the provision of the precursor material and the generation of the first radiation in the animal residence.

In particular, in embodiments, the method may comprise providing precursor material and first radiation in the animal residence, wherein the first radiation is selected (suitable) for converting the precursor material to olfactory molecules.

The method may especially comprise locally controlling animals in the animal residence, such as to control a subset of animals in the animal residence, especially to control a specific animal. In further embodiments, the method may comprise one or more of (i) locally providing the precursor material in the animal residence, and (ii) locally providing the first radiation in the animal residence.

In further embodiments, the animals may be selected from the group comprising poultry, cattle, pigs and aquatic animals.

In further embodiments, as described above, the olfactory molecules may comprise pheromones (for the animals). In yet further embodiments, the olfactory molecules may comprise scent molecules (for the animals).

In further embodiments, the method may comprise using the system or the animal residence system of the invention.

Similarly, in embodiments, the system, especially the control system, may be configured to execute (in an operational mode) the method of the invention.

The embodiments described herein are not limited to a single aspect of the invention. For example, an embodiment describing the method may, for example, further relate to the system, especially to an operational mode of the system, or especially to the control system. Similarly, an embodiment of the system describing an operation of the system may further relate to embodiments of the method. In particular, an embodiment of the method describing an operation (of the system) may indicate that the system may, in embodiments, be configured for and/or be suitable for the operation. Similarly, an embodiment of the system describing actions of (a stage in) an operational mode may indicate that the method may, in embodiments, comprise those actions.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1-2 schematically depict embodiments of the system.

The schematic drawings are not necessarily on scale.

### DETAILED DESCRIPTION OF THE EMBODIMENTS

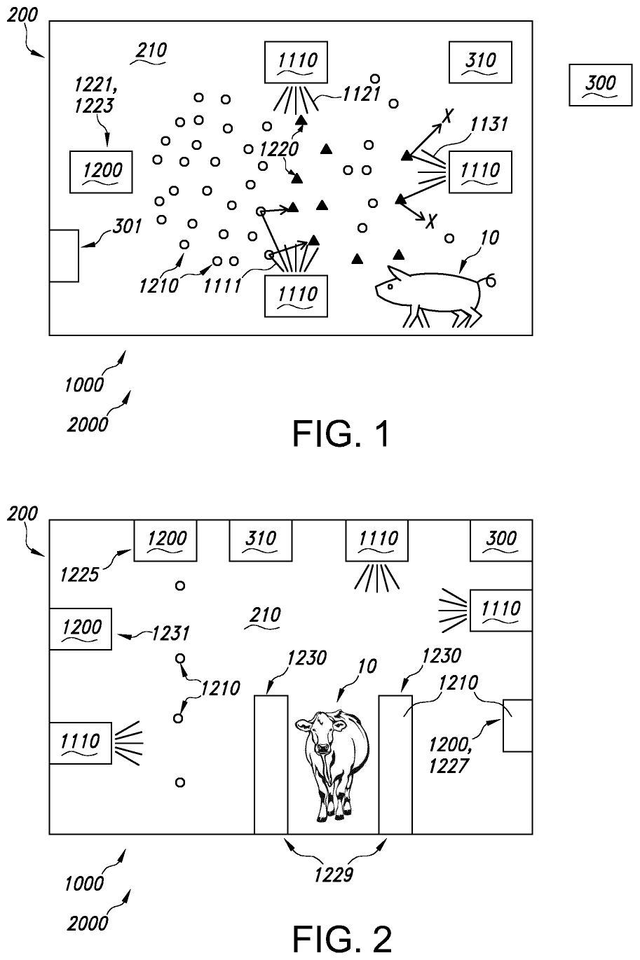

FIG. 1 schematically depicts an embodiment of the system 1000 for providing olfactory molecules 1220 in an animal residence 200. In the depicted embodiment, the system 1000 comprises a radiation system 1100, and a precursor material providing system 1200. In particular, the precursor material providing system 1200 may be configured to provide precursor material 1210. The precursor material 1210 may especially be (configured to be) at least partly convertible by first radiation 1111 into the olfactory molecules 1220. The radiation system 1100 may especially be configured to generate the first radiation 1111. Hence, as schematically depicted in FIG. 1, the first radiation 1111 may contact the precursor material 1210, triggering a conversion of the precursor material 1210 into the olfactory molecules 1220.

Hence, in embodiments, the precursor material 1210 and the radiation system 1100 may be configured to provide the olfactory molecules 1220 by converting at least part of precursor material 1210 into the olfactory molecules 1220 by irradiating the precursor material 1210 with the first radiation 1111

In embodiments, the system 1000 may further comprise a control system 300, especially wherein the control system 300 is configured to control generation of the olfactory molecules 1220 by controlling one or more of the radiation system 1100 and the precursor material providing system 1200. For instance, the control system 300 may be configured to control the precursor material providing system 1200 to (only) provide the precursor material 1210 when the olfactory molecules are desired, and/or to control the radiation system to (only) provide the first radiation 1111 when the olfactory molecules are desired.

In the depicted embodiment, the radiation system 1100 may be configured to generate first radiation 1111, second radiation 1121, and third radiation 1131. In particular, the first radiation 1111, especially a first wavelength of the first radiation 1111, may be selected for converting the precursor material 1210 to the olfactory molecules 1220. The second radiation 1121 may especially be visible radiation. The third radiation 1131 may especially be suitable for removal of the olfactory molecules 1220, such as by conversion of the olfactory molecules 1220 to a different molecule.

Hence, in embodiments, the radiation system 1100 may be configured to generate in a first operational mode (of the radiation system 1100) the first radiation 1111, and in a second operational mode (of the radiation system 1100) second radiation 1121, and in a third operational mode (of the radiation system 1100) third radiation 1131. Especially, the first radiation 1111, the second radiation 1121, and the third radiation may have different spectral power distributions. In particular, in embodiments, the first radiation 1111 may comprise UV radiation, the second radiation 1121 may comprise visible radiation, and the third radiation may be configured to modify the olfactory molecules 1220 to species that are not olfactory, have a smaller olfactory effect, or have a different olfactory effect.

In the depicted embodiment, the precursor material providing system 1200 may especially comprises a nebulizer system 1221, or a spray system 1223.

FIG. 1 further schematically depicts an embodiment of an animal residence system 2000. In particular, the animal residence system 2000 comprises the animal residence 200 and the system 1000 according to the invention. In embodiments, the radiation system 1100 may be configured to generate the first radiation 1111 in the animal residence 200, especially in an indoor space 210 of the animal residence 200. In further embodiments, the precursor material providing system 1200 may be configured to provide the precursor material 1210 in the animal residence 200, especially in the indoor space 210.

In further embodiments, the control system 300 may be configured to control generation of the olfactory molecules 1220 in the animal residence 200, especially in the indoor space 210, by controlling one or more of the radiation system 1100 and the precursor material providing system 1200.

In the depicted embodiment, the radiation system 1100 comprises a plurality of first light sources 1110 configured to provide the first radiation 1111 at the different locations in the animal residence 200. In particular, in the depicted embodiment, three first light sources 1110 are depicted in three different locations in the animal residence 200, wherein one of the three first light sources is depicted providing the first radiation 1111, one of the three first light sources is depicted providing the second radiation 1121, and one of the three light sources is depicted providing the third radiation 1131. Hence, in the depicted embodiment, the light sources may be configured to provide different types of radiation. However, in alternative embodiments, the first light sources 1110 may, for instance, be dedicated light sources for the first radiation 1111, and further (second and/or third) light sources may be arranged for providing the second and third radiation.

In embodiments, the control system 300 may be configured to control one or more of (i) a rate with which the olfactory molecules 1220 are provided, (ii) an amount in which the olfactory molecules 1220 are provided, and (iii) the type of olfactory molecules 1220 provided in dependence of one or more of (a) a user input, (b) a sensor signal of a sensor 310 (configured within the animal residence 200), and (c) a timer.

Hence, in embodiments, the control system 300 may comprise a user interface 301 configured to receive a user input.

In further embodiments, the system 1000 may comprise or be functionally coupled to a sensor 310, especially wherein the sensor 310 is arranged in the animal residence 200. In the depicted embodiment, the sensor may be configured to determine an animal behavioral parameter in the animal residence 200 and to provide a related behavior signal to the control system 300. The control system 300 may especially be configured to control the radiation system 1100 and/or the precursor material providing system 1200 in dependence of the behavior signal. For instance, the sensor 310 may be configured to detect aggressive behavior, such as based on one or more of movement and sound, or a behavior indicative of pending aggressive behavior, and may provide a related behavior signal to the control system 300, whereafter the control system 300 controls the generation of the olfactory molecules 1220 to reduce the aggressive behavior.

In embodiments, the animal residence 200 may be configured to one or more of poultry, cattle, pigs, and aquatic animals, such as fish, or such as crustaceans. In the depicted embodiment, the animal residence 200 may especially be configured to host pigs.

FIG. 1 further schematically depicts a method for controlling animals 10 in an animal residence 200. The method may comprise providing precursor material 1210 in the animal residence 200, wherein the precursor material 1210 is configured to be at least partly converted by first radiation 1111 into olfactory molecules 1220, especially wherein the olfactory molecules are olfactory molecules 1220 for one or more of the animals 10 in the animal residence 200. The method may further comprise controlling the generation of the olfactory molecules 1220 by controlling one or more of the provision of the precursor material 1210 and the generation of the first radiation 1111 in the animal residence 200.

In embodiments, the method may comprise one or more of (i) locally providing the precursor material 1210 in the animal residence 200, and (ii) locally providing the first radiation 1111 in the animal residence 200. Thereby, especially a subset of animals 10 in the animal residence 200 may be controlled, such as especially a single animal 10 in the animal residence 200.

FIG. 2 schematically depicts a further embodiment of the system 1000 and of the animal residence system 2000.

In the depicted embodiment, the precursor material providing system comprises a dripping system 1225, a first radiation accessible precursor material reservoir 1227, an applicator system 1229 (for (targeted) application of the precursor material 1210 on an animal 10), and a fluid flow control system 1231.

Hence, in the depicted embodiment, the precursor material providing system 1200 may be configured to provide the precursor material 1210 at different locations in the animal residence 200. Similarly, in the depicted embodiment, the radiation system 1100 may be configured to provide the first radiation 1111 at the different locations in the animal residence 200, especially by controlling which of the first light sources 1110 provide the first radiation 1111. In further embodiments, the control system 300 may be configured to control local generation of the olfactory molecules 1220 in the animal residence 200 by controlling one or more of (i) the precursor material providing system 1200 and (ii) the radiation system 1100, especially the first light sources 1110 of the radiation system 1100.

In particular, with respect to the first radiation 1111 accessible precursor material reservoir 1227, the accessible precursor material reservoir 1227 may especially be configured to passively provide the precursor material 1210 (at a specific location). For instance, the accessible precursor material reservoir 1227 may comprise an adhesive element, such as a sticker, arranged on a wall of the animal residence 200. In further embodiments, the precursor material 1210 and the radiation system 1100 may be configured to release the olfactory molecules 1220 from the precursor material 1210 by irradiating the precursor material 1210 with the first radiation 1111.

With respect to the applicator system 1229, the applicator system may comprise one or more applicator system elements 1230 configured to apply the precursor material 1210 to an animal. In the depicted embodiment, the applicator system elements 1230 may, for instance, comprise (comfort) brushes configured to apply the precursor material 1210 on the animal 10 as the animal 10 brushes against the brushes.

In further embodiments, one or more of the following may apply: (i) a position of at least one applicator system element 1230 within the animal residence 200 is controllable by the control system 300; (ii) the animal residence system 2000 comprises a plurality of applicator system elements 1230 configured at different locations within the animal residence 200 and configured to locally apply the precursor material 1210 to the animal; and (iii) at least one applicator system element 1230 is configured at a location where the animal is at least at a predefined frequency, such as at least once a day.

The term “plurality” refers to two or more. Furthermore, the terms “a plurality of” and “a number of” may be used interchangeably.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment, but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A system for providing olfactory molecules in an animal residence, the system comprising (a) a radiation system, (b) a precursor material providing system, and (c) a control system, wherein:
the radiation system is configured to generate first radiation, wherein the radiation system comprises one or more light sources;
the precursor material providing system is configured to provide precursor material, wherein the precursor material is configured to be at least partly converted by the first radiation into the olfactory molecules; and
the control system is configured to control generation of the olfactory molecules by controlling one or more of the radiation system and the precursor material providing system.

2. The system according to claim 1, wherein the radiation system is configured to generate in a first operational mode the first radiation and in a second operational mode second radiation, wherein the first radiation and the second radiation have different spectral power distributions; wherein the first radiation comprises UV radiation and wherein the second radiation comprises visible radiation.

3. The system according to claim 1, wherein the precursor material providing system comprises one or more of a (i) nebulizer system, (ii) a spray system, (iii) a dripping system, (iv) a first radiation accessible precursor material reservoir, (v) an applicator system, and (vi) a fluid flow control system.

4. The system according to claim 1, wherein the precursor material and the radiation system are configured to release the olfactory molecules from the precursor material by irradiating the precursor material with the first radiation and/or wherein the precursor material and the radiation system are configured to provide the olfactory molecules by converting at least part of precursor material into the olfactory molecules by irradiating the precursor material with the first radiation.

5. The system according to claim 1, wherein the radiation system comprises a plurality of first light sources configured to provide the first radiation at different locations in the animal residence.

6. An animal residence system comprising (i) an animal residence and (ii) the system according to claim 1, wherein:
the radiation system is configured to generate the first radiation in the animal residence);
the precursor material providing system is configured to provide the precursor material in the animal residence; and
the control system is configured to control generation of the olfactory molecules in the animal residence by controlling one or more of the radiation system and the precursor material providing system.

7. The animal residence system according to claim 6, wherein:
the precursor material providing system is configured to provide the precursor material at different locations in the animal residence;
the radiation system is configured to provide the first radiation at the different locations in the animal residence, wherein the radiation system comprises a plurality of first light sources configured to provide the first radiation at the different locations in the animal residence; and
the control system is configured to control local generation of the olfactory molecules in the animal residence by controlling one or more of (i) the precursor material providing system and (ii) the radiation system.

8. The animal residence system according to claim 7, wherein the precursor material providing system comprises the applicator system, wherein the applicator system comprises one or more applicator system elements configured to apply the precursor material to an animal; wherein one or more of the following applies: (i) a position of at least one applicator system element within the animal residence is controllable by the control system; (ii) the animal residence system comprises a plurality of applicator system elements configured at different locations within the animal residence and configured to locally apply the precursor material to the animal; and (iii) at least one applicator system element is configured at a location where the animal is at least once a day.

9. The animal residence system according to claim 6, wherein the control system is configured to control one or more of (i) a rate with which the olfactory molecules are provided, (ii) an amount in which the olfactory molecules are provided, and (iii) the type of olfactory molecules provided in dependence of one or more of (a) a user input, (b) a sensor signal of a sensor, (c) a timer.

10. The animal residence system according to claim 9, wherein the animal residence system comprises the sensor, wherein the sensor is configured to determine an animal behavioral parameter in the animal residence and to provide a related behavior signal to the control system, wherein the control system is configured to control the radiation system and/or the precursor material providing system in dependence of the behavior signal.

11. The animal residence system according to claim 6, wherein:
the radiation system is configured to generate in a first operational mode the first radiation;
the radiation system is configured to generate in a second operational mode second radiation;
the radiation system is configured to generate in a third operational mode third radiation;
the first radiation, the second radiation, and the third radiation have different spectral power distributions;
the first radiation comprises UV radiation;
the second radiation comprises visible radiation; and
the third radiation is configured to modify the olfactory molecules to species that are not olfactory, have a smaller olfactory effect, or have a different olfactory effect.

12. The animal residence system according to claim 6, wherein the animal residence is configured to host insects, poultry, cattle, pigs, or aquatic animals.

13. A method for controlling animals in an animal residence, the method comprising:
providing precursor material in the animal residence, wherein the precursor material is configured to be at least partly converted by first radiation into at least olfactory molecules, wherein the olfactory molecules are olfactory molecules for one or more of the animals in the animal residence; and
controlling the generation of the olfactory molecules by controlling one or more of the provision of the precursor material and the generation of the first radiation in the animal residence.

14. The method according to claim 13, wherein the method comprises one or more of (i) locally providing the precursor material in the animal residence, and (ii) locally providing the first radiation in the animal residence.

15. The method according to claim 13, wherein the animals are selected from the group comprising insects, poultry, cattle, pigs and aquatic animals, and wherein the olfactory molecules comprise pheromones.
